(19) United States
(12) Patent Application Publication
(10) Pub. No.: US 2021/0179988 A1
(43) Pub. Date: Jun. 17, 2021

(54) DIGESTER COMPRISING AN INNER WALL HAVING EXPANSIONS AND/OR HOLLOWS

(71) Applicant: L'Air Liquide, Societe Anonyme pour l'Etude et l'Exploitation des Procedes Georges Claude, Paris (FR)

(72) Inventors: Aude BERTRANDIAS, Les Loges-En-Josas (FR); David FRIMAT, Les Loges-En-Josas (FR); Antonio TRUEBA, Les Loges-En-Josas (FR); Jacopo SEIWERT, Les Loges-En-Josas (FR); Jeremy OLLIER, Sassennage (FR); Solene VALENTIN, Sassennage (FR)

(73) Assignee: L'Air Liquide, Societe Anonyme pour l'Etude et l?Exploitation des Procedes Georges Claude, Paris (FR)

(21) Appl. No.: 17/118,494

(22) Filed: Dec. 10, 2020

(30) Foreign Application Priority Data
Dec. 11, 2019 (FR) FR 1914169

(51) Int. Cl. C12M 1/107; C12P 5/02; B01D 53/85; B01D 53/52

(52) U.S. Cl. CPC C12M 21/04; C12P 5/023; B01D 2251/11; B01D 53/52; B01D 2251/102; B01D 53/85

(57) ABSTRACT

Plant for producing at least partially desulfurized biogas, comprising a biomass digester and/or post-digester, the digester and/or post-digester comprising:
a chamber comprising the biomass and the gas space, and
a means for introducing an oxidizing gas,

characterized in that the portion of the inner wall of the chamber situated at the level of the gas space exhibits expansions and/or hollows.

### CROSS-REFERENCE TO RELATED APPLICATIONS

This application claims the benefit of priority under 35 U.S.C. § 119 (a) and (b) to French patent application No. FR 1914169, filed Dec. 11, 2019, the entire contents of which are incorporated herein by reference.

### FIELD OF THE INVENTION

The present invention relates to a plant and a process for producing at least partly desulfurized biogas.

### BACKGROUND

Biogas is the gas produced during the decomposition of organic matter in the absence of oxygen (anaerobic fermentation), also known as methanisation. The decomposition may be natural, as observed in swamps or in household rubbish dumps, though the production of biogas may also result from the methanisation of wastes in a dedicated reactor, under controlled conditions, known as a methaniser or digester, and then in a post-digester, which is similar to the digester and allows the methanisation reaction to be extended.

Biomass refers to any group of organic matters that can be converted into energy through this methanisation process: for example, treatment plant sludges, manures/slurries, agricultural residues, food wastes, etc.

The digester, namely the A reactor dedicated to the methanisation of the biomass, is a closed vessel, heated or not (operated at a set temperature, between the ambient temperature and 55° C.), the contents of said vessel, composed of the biomass, being mixed, continuously or sequentially. The conditions in the digester are anaerobic, and the biogas generated is found in the headspace of the digester (gas space), from where it is withdrawn. Post-digesters are similar to digesters.

Owing to its main constituents—methane and carbon dioxide—biogas is a powerful greenhouse gas; at the same time, it also constitutes a source of renewable energy, which is appreciable in the context of the increasing scarcity of fossil fuels.

Biogas contains predominantly methane (CH₄) and carbon dioxide (CO₂), in proportions which can vary according to the substrate and to the way in which the biogas is obtained; it may also contain, in smaller proportions, water, nitrogen, hydrogen sulfide (H₂S), oxygen, and also other organic compounds, in the form of traces, including H₂S, between 10 and 50 000 ppmv.

Depending on the organic matter which has undergone decomposition, and on the techniques used, the proportions of the components differ; on average, however, biogas comprises, on a dry gas basis, from 30% to 75% of methane, from 15% to 60% of CO₂, from 0% to 15% of nitrogen, and from 0% to 5% of oxygen and trace compounds.

Biogas is made use of economically in various ways. It can, after a gentle treatment, be exploited close to the production site in order to supply heat, electricity or a mixture of both (cogeneration); the high carbon dioxide content reduces its calorific value, increases the costs of compression and of transportation and limits the economic advantage of making use of it economically to this use nearby.

More intensive purification of biogas allows it to be more widely used; in particular, intensive purification of biogas makes it possible to obtain a biogas which has been purified to the specifications of natural gas and which can be substituted for the latter; biogas thus purified is known as “biomethane”. Biomethane thus supplements natural gas resources with a renewable part produced within territories; it can be used for exactly the same uses as natural gas of fossil origin. It may supply a natural gas network or a vehicle filling station; it may also be liquefied for storage in the form of liquefied natural gas (bioLNG), etc.

Depending on the composition of the biomass, the biogas produced in the digestion contains hydrogen sulfide (H₂S) in amounts of between 50 and 50 000 ppm.

Irrespective of the final commercial destination of the biogas, it proves to be vital to remove hydrogen sulfide, which is a toxic and corrosive impurity. Moreover, if the use of the biogas involves purifying it for injection of biomethane into the natural gas network, there are strict specifications limiting the permitted quantity of H₂S.

A number of methods exist for removing H₂S and are more or less widespread (beds of activated carbon, addition of iron compounds, physical or chemical absorption, water washing, biofilters, etc.). Removal is accomplished primarily by adsorption on a bed of activated carbon, outside the digester. In an increasing number of digesters, H₂S abatement is accomplished in part by injecting air/enriched air/O₂ into the gas space of the digester, so constituting an in situ solution. With injection into the gas space at a low rate, solid sulfur is formed from the H₂S and O₂ (eq. (1)), as performed by sulfur-oxidizing bacteria, e.g. Thiobacillus. With a high rate of O₂ injected, the mixture is acidified (eq. (2)). The target reaction is therefore reaction (1).

H₂S+0.5O₂→S+H₂O (1)

H₂S+2O₂→SO₄²⁻+2H+ (2)

The amounts of O₂ which need to be injected in practice are different from those expected from the stoichiometry of eq. (1): doses of 0.3%-3% O₂ relative to the biogas generated are most usually recommended, with doses of up to 12% being sometimes stated.

Presently, the in situ injection of air/enriched air/O₂ is not optimized, and the beds of activated carbon must therefore be maintained in order to remove all of the H₂S.

From this basis, one problem which arises is that of providing an improved plant promoting greater removal of H₂S.

### SUMMARY

A solution of the present invention is a plant for producing at least partially desulfurized biogas, comprising a biomass digester and/or post-digester, the digester and/or post-digester comprising:
a chamber comprising the biomass and the gas space, and
a means for introducing an oxidizing gas,

characterized in that the portion of the inner wall of the chamber situated at the level of the gas space exhibits expansions and/or hollows.

Preferably, the expansions and the hollows enable a multiplication by at least 1.5, preferably at least 2, more preferably at least 3 times of the “standard” surface area of the portion of inner wall of the chamber that is situated at the gas space. A “standard” surface area refers to a smooth surface, namely without any expansion and/or hollows.

“Gas space” refers to the space in the digester or post-digester that contains gas (as opposed to the space which contains the liquid).

### BRIEF DESCRIPTION OF THE FIGURES

For a further understanding of the nature and objects for the present invention, reference should be made to the following detailed description, taken in conjunction with the accompanying drawings, in which like elements are given the same or analogous reference numbers and wherein:

FIG. 1 is a horizontal schematic section through the chamber of the digester.

### DETAILED DESCRIPTION OF THE INVENTION

The reactions between O₂ and H₂S take place on a surface (not in the gaseous or liquid phase of the digester). The microorganisms needed for the reaction (sulfur-oxidizing bacterial such as Thiobacillus) are in the liquid phase, and the reagents are in the gas phase. The hydrophilic surface is required for contact between the liquid phase, the microorganisms, and the gas phase, the reagents. These surfaces are primarily the inner walls in the gas space, and the gas/liquid interface of the digester or post-digester. If the surface areas available are insufficient, the H₂S abatement reactions do not take place sufficiently, or even not all.

Expressed alternatively, the solution according to the invention, by increasing the surface area of the inner wall of the chamber, enables an increase in the size of the reaction support and so promotes the more advanced removal of hydrogen sulfide.

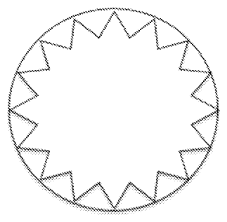

Depending on the case, the plant according to the invention may have one or more of the features below:
the expansions project towards the interior of the chamber,
the expansions project radially,
the expansions and/or the hollows are in succession along the perimeter of the chamber,
the portion of inner wall of the chamber situated at the level of the gas space exhibits a zigzag shape; this zigzag shape is shown in FIG. 1, which is a diagrammatic horizontal section through the chamber of the digester or post-digester. Note that the section is made in the portion of the inner wall of the chamber situated at the level of the gas space.
the inner wall is composed of a material which is resistant to a humid and corrosive atmosphere; examples might include certain stainless steels, PEEK, PTFE,
the inner wall is divided into a first wall and a second wall, the two walls being side by side, and the second wall being opposite the interior of the chamber and having a surface area greater than the surface area of the first wall.

A further subject of the present invention is a process for producing at least partially desulfurized biogas, using a plant according to the invention, which comprises:
injecting biomass into the digester;
injecting an oxidizing gas at the top of the digester; and
mixing the biomass.

Note that the oxidizing gas might be oxygen or air or enriched air. Enriched air refers to air having a higher oxygen content than the oxygen content normally present in air.

Inside the digester, when hydrogen sulfide reacts with oxygen, the sulfur attaches to the inner wall of the chamber of the digester. After a certain time, the solid sulfur generated falls into the digestate and is removed with said digestate. The solution according to the invention allows a biogas stream to be obtained that comprises less than 200 ppm of hydrogen sulfide.

The invention makes it possible to reduce the costs of purifying biogas by removal of hydrogen sulfide effectively, by increasing the reactivity of the oxygen already injected with the sulfur-containing products, by creating an additional reaction surface on the inner walls of the digester at the level of the gas space, with no need for complex engineering.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing i.e. anything else may be additionally included and remain within the scope of “comprising.” “Comprising” is defined herein as necessarily encompassing the more limited transitional terms “consisting essentially of” and “consisting of”; “comprising” may therefore be replaced by “consisting essentially of” or “consisting of” and remain within the expressly defined scope of “comprising”.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

While embodiments of this invention have been shown and described, modifications thereof may be made by one skilled in the art without departing from the spirit or teaching of this invention. The embodiments described herein are exemplary only and not limiting. Many variations and modifications of the composition and method are possible and within the scope of the invention. Accordingly the scope of protection is not limited to the embodiments described herein, but is only limited by the claims which follow, the scope of which shall include all equivalents of the subject matter of the claims.

1. A plant for producing at least partially desulfurized biogas, comprising a biomass digester and/or post-digester, the digester and/or post-digester comprising:
a chamber comprising the biomass and the gas space, and
a means for introducing an oxidizing gas,
characterized in that the portion of the inner wall of the chamber situated at the level of the gas space exhibits expansions and/or hollows.

2. The plant of claim 1, wherein the expansions project towards the interior of the chamber.

3. The plant of claim 2, wherein the expansions project radially.

4. The plant of claim 1, wherein the expansions and/or the hollows are in succession along the perimeter of the chamber.

5. The plant of claim 1, wherein the portion of inner wall of the chamber situated at the level of the gas space exhibits a zigzag shape.

6. The plant of claim 1, wherein the inner wall is composed of a material which is resistant to a humid and corrosive atmosphere.

7. The plant of claim 1, wherein the inner wall is divided into a first wall and a second wall, the two walls being side by side, and the second wall being opposite the interior of the chamber and exhibiting a surface area greater than the surface area of the first wall.

8. Process for producing desulfurized biogas, using a plant according to claim 1, which comprises:
injecting biomass into the digester;
injecting an oxidizing gas at the top of the digester; and
mixing the biomass.

9. The process of claim 8, wherein the biogas is mixed so as to promote transfer of the oxidizing gas to the inner wall of the chamber.

10. The process of claim 8, wherein the oxidizing gas is oxygen or air or enriched air.
